(19) United States
(12) Patent Application Publication
(10) Pub. No.: US 2022/0107301 A1
(43) Pub. Date: Apr. 7, 2022

(54) SYSTEM FOR THE MEASUREMENT OF RHEOLOGICAL PROPERTIES OF A FLUID

(71) Applicant: Abductive Services LLC, Bryan, TX (US)

(72) Inventor: Paul Chike Ofoche, Bryan, TX (US)

(73) Assignee: Abductive Services LLC, Bryan, TX (US)

(21) Appl. No.: 17/111,034

(22) Filed: Dec. 3, 2020

Related U.S. Application Data
(63) Continuation-in-part of application No. 17/063,903, filed on Oct. 6, 2020.

(51) Int. Cl. G01N 33/28; G01N 11/06; G01N 9/00

(52) U.S. Cl. CPC G01N 33/2823; G01N 9/00; G01N 11/06

(57) ABSTRACT

The present disclosure provides a system for the measurement of rheological properties of a fluid including a computing device including memory, one or more processors, and software executable by the one or more processors, configured to accept input parameters including a fluid density and the fluid viscometer drain time; and subsequently output one or more values corresponding to one or more rotations per minute (RPM) dial readings (and in centipoise) of a conventional rheometer. In embodiments, the input parameters may be limited to, or consist of, fluid density and fluid viscometer drain time. In embodiments, the system may output and display six values corresponding to the six RPM dial readings (and in centipoise) of a conventional rheometer corresponding to 600 RPM, 300 RPM, 200 RPM, 100 RPM, 6 RPM, and 3 RPM.

### CROSS REFERENCE TO RELATED APPLICATIONS

This application is a continuation in part of U.S. application Ser. No. 17/063,903, filed on Oct. 6, 2020, which is herein incorporated by reference in its entirety.

### BACKGROUND OF THE DISCLOSURE

### Field of the Invention

This disclosure generally relates to a computing device comprising software configured to accept fluid viscometer input measurements and parameters and then output data corresponding to conventional rheometer dial readings (and in centipoise) at various rotations per minute (RPM).

### Description of the Related Art

There is a need to frequently and more easily measure fluid rheological properties accurately without the requirement to run sophisticated laboratory equipment. Current state-of-the-art in rheometer technology makes use of sophisticated laboratory scale equipment which is not readily available or frequently utilized during industry operations and field processes. Currently available technology to measure fluid rheological properties requires time to operate and analyze the rheology measurements which are infrequently obtained. Existing technology is also not capable of reporting rheological readings under different flow conditions. A conventional rotational rheometer must be operated each time to obtain measurements of fluid rheology. Thus, proper monitoring of fluid rheology in a frequent manner is not possible using current state-of-the-art technology.

Thus, there is a need for devices including software that readily plot and display rheological properties graphically under different flow conditions based on simple inputs of fluid density and viscometer funnel drain times that can be measured in the field. This would simplify the monitoring of fluid rheology and help to ensure the proper monitoring and measurement of fluid rheological profiles. It would also address a need to make rheology reports instant and more frequently obtained.

### SUMMARY OF THE INVENTION

This invention is directed to devices and software applications to report and display the readings of fluid rheology under different flow conditions simultaneously. The readings reported are equivalent to those obtained from conventional rotational rheometers which are currently the industry standard.

This disclosure is accordingly directed to mobile phones, tablets, computers, graphical and visual mobile display units, dashboards, etc., including software that can, in one embodiment, take two (2) input values, i.e. fluid density and drain time and output rheological readings which are equivalent to dial readings (and in centipoise) from conventional direct-indicating rotational rheometers. The two input values, i.e., fluid density and drain time may be obtained, for example, from a viscometer funnel including a Marsh funnel or Marsh cone or a goblet viscometer as disclosed, for example, in parent U.S. application Ser. No. 17/063,903, titled GOBLET VISCOMETER.

The system then outputs dial readings (and in centipoise) at several rotational speeds (3-600 RPM) or corresponding shear rates which would equivalently be obtained from a conventional 6-speed rheometer. This invention can additionally display the multiple readings in a graph, thereby making it easy for users to visualize the rheological properties of fluids. Other derivative values for describing fluid rheology, such as yield point, plastic viscosity and apparent viscosity can also be provided by the system.

The present invention thus provides for a system capable of taking two simple and easily obtained or measured input values, i.e., fluid density and funnel viscometer drain time (e.g., a marsh funnel or a goblet viscometer) and outputting in real time dial readings (and in centipoise) at one or more rotational speeds (RPM) which would be equivalently obtained from a conventional 6 speed rheometer.

The present invention thus measures and reports rheological readings under different flow conditions in real time. As mentioned, a conventional rotational rheometer must be operated separately each time to obtain measurements of fluid rheology. Thus, proper monitoring of fluid rheology in a frequent manner, in real time, is not possible. The disclosed system includes mobile computing devices including software that readily output and display fluid rheological properties graphically under different flow conditions based on simple inputs of fluid density and drain time that can be measured in the field. This simplifies the monitoring of fluid rheology and helps to ensure the proper monitoring and measurement of fluid rheological profiles. It also addresses the need to make rheology reports instant and more readily obtained, particularly in the field.

Accordingly, the present disclosure describes a system for the measurement of rheological properties of a fluid, including a computing device including memory, one or more processors, and software executable by the one or more processors, configured to accept input parameters including a fluid density and a fluid viscometer drain time; and subsequently outputs one or more values corresponding to one or more rotations per minute (RPM) dial readings (and in centipoise) of a conventional rheometer.

In embodiments, the input parameters may be limited to, or consist of, fluid density and fluid viscometer drain time. In embodiments, the system may output 6 values corresponding to the 6-speed dial readings (and in centipoise) of a conventional rheometer corresponding to 600 RPM, 300 RPM, 200 RPM, 100 RPM, 6 RPM, and 3 RPM.

Other features and aspects will be apparent from the following detailed description, the drawings, and the claims

### BRIEF DESCRIPTION OF THE DRAWINGS

FIG. 1 shows one embodiment of an output reading display for six RPM dial readings of a conventional rheometer.

FIG. 2 shows one embodiment of an output reading display showing plastic viscosity and yield point.

FIG. 3 shows one embodiment of the operation of the software application of the disclosure.

FIG. 4 shows a feature importance of the output dial readings calculation input parameters.

FIG. 5 shows feature importance of machine learning process input parameters.

FIG. 6 shows a depiction of a tree from an Extreme Gradient Boosted Trees model.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

One important aspect of this invention that distinguishes it from existing technology is its ease of use. Simply entering two input values of density and drain time produces multiple values that would be obtained from a conventional rotational rheometer. The invention can be utilized on mobile phones, tablets, computers, graphical and visual display units, vehicle dashboard computing and display units, etc.

The existing technology is not capable of reporting rheological readings under different flow conditions in real time. A conventional rotational rheometer would have to be operated separately each time to obtain measurements of fluid rheology. This invention simplifies the process by making the rheological values readily available based on only two inputs. This invention can serve as an important resource in operational environments, such as the petroleum industry, food processing industry, cement industry, etc. where frequent monitoring and measurement of fluid rheology is required.

This invention can be implemented on various hardware, including but not limited to mobile phones and devices, tablets, laptop computers, desktop computers, graphical and visual display units, vehicle computing units in dashboards, etc. The primary output readings obtained are the 3, 6, 100, 200, 300 and 600 RPM (rotations per minute) dial readings (and in centipoise) equivalent to those of a conventional rotational rheometer, plastic viscosity, yield point, and apparent viscosity, as well as a graph showing all these values. Additional values of choice can also be displayed.

The invention uses two input parameters, density and viscometer drain time, and using artificial intelligence techniques, including machine learning and/or neural networks, calculates and outputs conventional rheometer RPM dial readings (and in centipoise). In embodiments, the machine learning techniques include gradient boosting, gradient tree boosting (boosted trees), bootstrap forests, or other like algorithms.

The invention uses some or all of the following parameters and variables as predictors to arrive at predicted conventional dial readings (and in centipoise) for each RPM: nominal height, wall shear, pressure differential, flow coefficient, mass flow rate, Graham's Law of diffusion (gases), and relative change in viscosity with square root of shear rate. These parameters are further described below.

Nominal height=l, which is the height determined by interpolation at which corresponding shear rates are obtained in a conventional rheometer.

Nominal height uses maximum shear rate=0 s⁻¹ and OFI model 900 shear rate at the particular RPM of interest (s⁻¹); Maximum shear=77115.77562/l³×Funnel Viscosity.

Funnel/Goblet time (s)=Time for draining 1 quart of fluid from the funnel.

Funnel/Goblet time delta (s)=Difference in time between the duration for draining 1 quart of any fluid and a completely inviscid fluid from the funnel.

$$Wall \text{shear:} {τ}_{w} = \frac{[ ρ g ( Z ) + {Z}_{2} ]}{\frac{Z}{cosα \{ {R}_{L} + ( {R}_{O} - {R}_{L} ) \frac{Z}{{Z}_{1}} \}} + \frac{2 {Z}_{2}}{{R}_{L}}}$$

$$Pressure \text{differential:} Δ P = {Δ P}_{cone} + {Δ P}_{cylinder} = \frac{{τ}_{w} Z}{cosα \{ {R}_{L} + ( {R}_{O} - {R}_{L} ) ( \frac{Z}{{Z}_{1}} ) \}} + \frac{2 {Z}_{2}}{{R}_{L}}$$

$$Flow \text{coefficient:} {C}_{v} = Flowrate \times \sqrt{\frac{Specific Gravity}{Pressure Drop}}$$

$$Mass Flow \text{Rate:} Mass Flow rate = Density \times \frac{1}{funnel time}$$

$$Mass flow rate = \frac{lb}{gal} \times \frac{qt}{sec} = \frac{gal}{4 \times sec} = \frac{lb}{sec}$$

$$\frac{Mass}{Time} = \frac{Mass}{{Length}^{3}} \times \frac{{Length}^{3}}{Time} = \frac{Mass}{Time}$$

$$Graham ' s law of diffusion \text{(gases):} \frac{{rate}_{1}}{{rate}_{2}} ∝ \sqrt{\frac{{density}_{2}}{{density}_{1}}}$$

Root density=Square root of density.

Relative change in viscosity with square root of shear rate:

Funnel Viscosity² ∝ Shear rate.

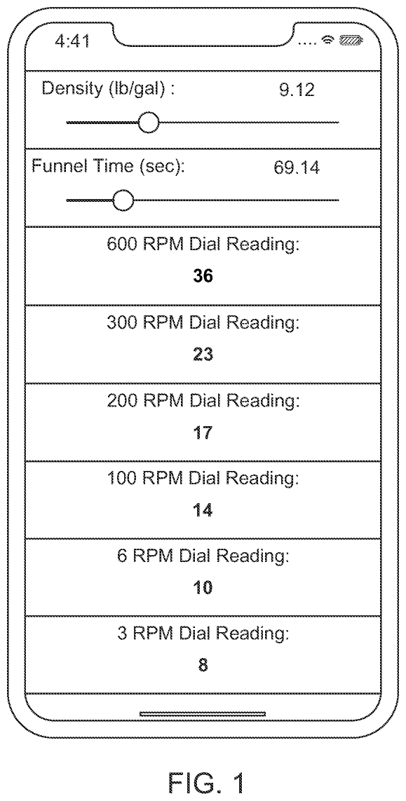

FIG. 1 shows one embodiment of an output reading display for six RPM dial readings of a conventional rheometer.

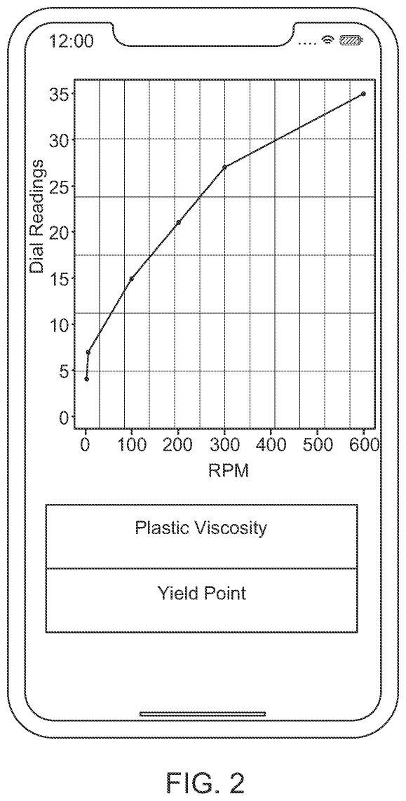

FIG. 2 shows one embodiment of an output reading display showing plastic viscosity and yield point.

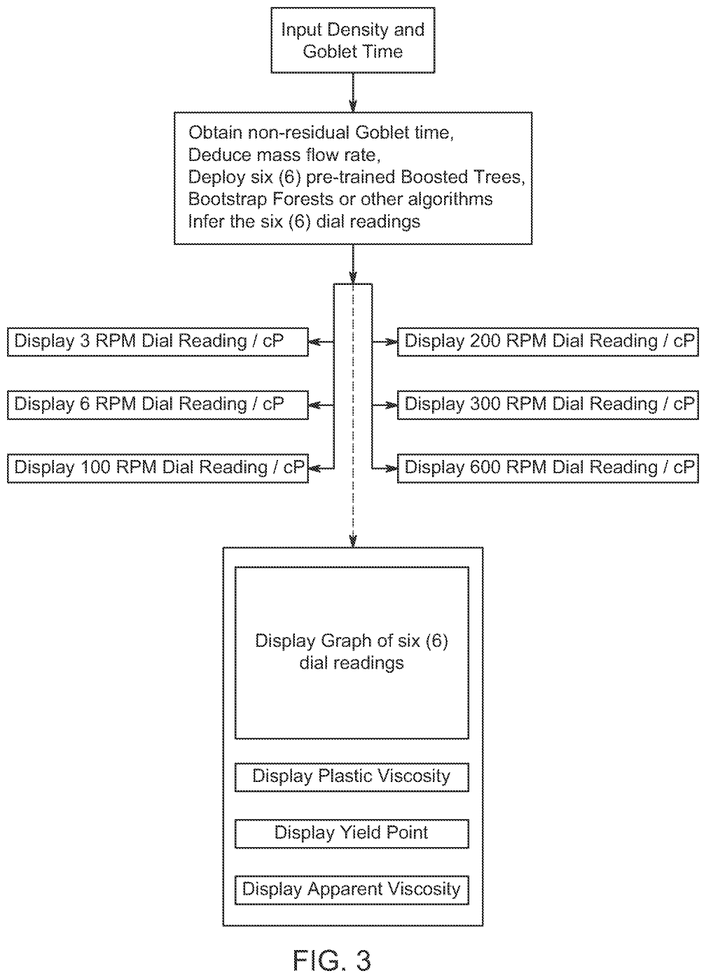

One embodiment of the operation of the software application is shown in FIG. 3. FIG. 3 shows input density and viscometer drain time as input parameters. The output dial readings are obtained as shown. Next, non-residual drain time is obtained. The mass flow rate is then deduced. Six pre-trained boosted trees are then deployed. Bootstrapped forests or other algorithms then calculate six RPM dial readings (and in centipoise) equivalent to those of a conventional rotational rheometer. Funnel time and density are the only two inputs required.

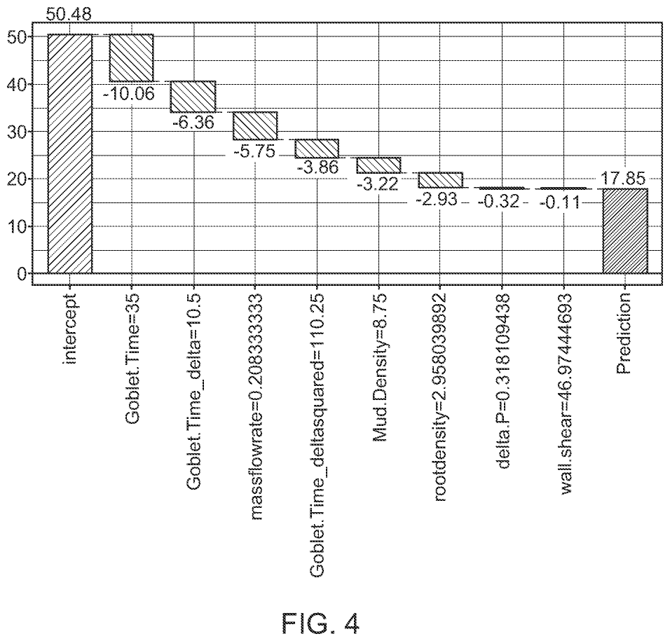

FIG. 4 shows a feature importance of input parameters for an output conventional dial readings calculation. With goblet funnel time or Marsh funnel time and density being identified as the most important features.

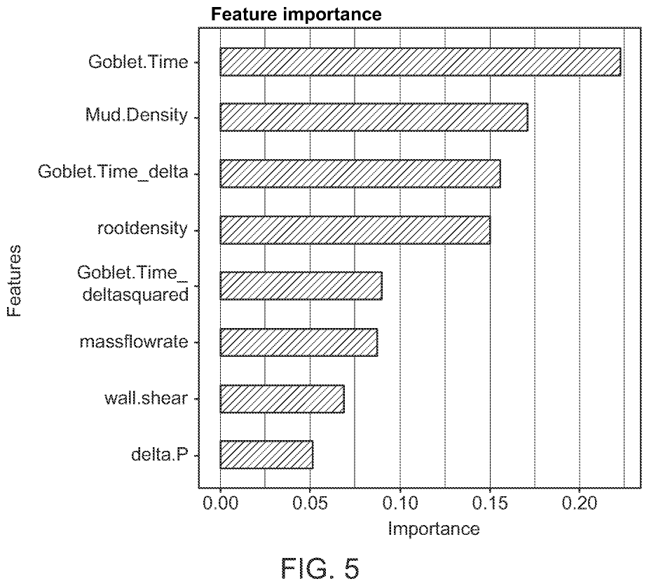

FIG. 5 shows feature importance of the machine learning process input parameters. With the Goblet or Marsh Funnel Time and Density being identified as the most important features.

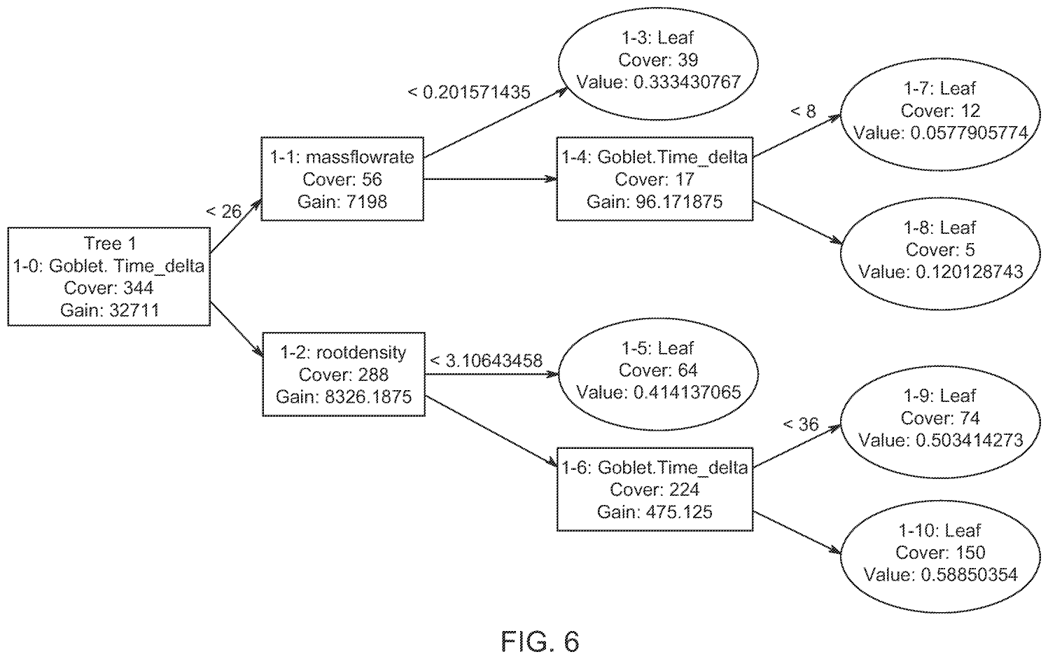

FIG. 6 shows a depiction of a tree from an Extreme Gradient Boosted Trees model. Each node is binary, and a decision is made regarding the split path based upon a threshold value as indicated in the figure.

In embodiments, the invention can be implemented on various devices, such as, a personal computer, a mobile computing device, a notebook computer, a netbook, a mobile multifunction computing device, a personal digital assistant, a tablet computer, a mobile phone, a smart phone, etc.

The invention can thus be implemented in digital electronic circuitry, or in computer hardware, firmware, software, or in one or more combinations. Apparatus of the invention can be implemented in a computer program product tangibly embodied in a machine-readable storage device for execution by a programmable processor; and method steps in the invention can be performed by a programmable processor execution a program of instructions to perform functions of the invention by operating on input data and generating output. The invention can be implemented advantageously in one or more computer programs that are executable on a programmable system including at least one programmable processor coupled to receive data and instructions from, and to transmit data and instructions to, a data storage system, at least one input device, and at least one output device. Each computer program can be implemented in a high-level procedural or object-oriented programming language, or in assembly or machine language if desired; and in any case, the language can be a compiled or interpreted language.

Suitable processors include both general and special purpose microprocessors. Generally, a processor will receive instructions and data from a read-only memory and/or a random-access memory. Generally, a computer will include one or more storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and optical disks. Any of the foregoing can be supplemented by, or incorporated in, ASICs (application-specific integrated circuits).

A computer system may include a bus, a processor, a system memory, a read-only memory, a storage device, input devices, and output devices. In some embodiments, the computer system also includes a graphic processing unit.

The bus collectively represents system, peripheral, and chipset buses that support communication among internal devices of the computer system. For instance, the bus communicatively connects the processor with the read-only memory, the system memory, and the storage device.

From these various memory units, the processor (also referred to as a central processing unit or CPU) retrieves instructions to execute and data to process in order to execute the processes of the invention. The read-only-memory (ROM) stores static data and instructions that are needed by the processor and other modules of the computer system.

The storage device, on the other hand, is a read-and-write memory device. This device is a non-volatile memory unit that stores instructions and data even when the computer system is off. Some embodiments of the invention use a mass-storage device (such as a magnetic or optical disk and its corresponding disk drive) as a permanent storage device. The permanent storage device may be a fully solid-state storage, a conventional spinning magnetic pallet storage (i.e. a hard drive), or combinations thereof.

Other embodiments may use a removable storage device (such as a USB flash drive or SD Memory Card) as a temporary storage or as the permanent storage device.

Like the permanent storage device, the system memory is a read and write memory device. However, unlike a storage device, the system memory is a volatile read-and-write memory, such as a random-access memory. The system memory stores at least some of the instructions and data that the processor uses at runtime.

Instructions and/or data needed to perform processes of embodiments of the invention are stored in the system memory, the permanent storage device, the read-only memory, or any combination. For example, the various memory units may contain instructions for processing multimedia items in accordance with some embodiments. From these various memory units, the processor retrieves instructions to execute and data to process to execute the processes of the invention.

The bus also connects to input and output devices. The input devices enable the user to communicate information and select commands to the computer system. The input devices include alphanumeric keyboards, touch panels, and cursor controllers. The input devices also include scanners through which an image can be input to the computer system. The output devices display images generated by the computer system. The output devices may include printers, pen plotters, laser printers, ink-jet plotters, film recorders, and display devices, such as cathode ray tubes (CRT), liquid crystal displays (LCD), OLED's, or electroluminescent displays.

The bus may also connect a computer to a network. In this manner, the computer can be a part of a network of computers (such as a local area network (LAN), a wide area network (WAN), or an Intranet) or a network of networks (such as the Internet). Finally, the computer system in some embodiments also optionally includes a graphics processing unit (GPU). A GPU (also referred to as a visual processing unit or a display processor) is a dedicated graphics rendering device which can manipulate and display computer graphics. The GPU can be included in a video card or can be integrated into the mother board of the computer system along with the processor. Also, the computer system may be used as a personal computer, a workstation, a game console, or the like. Any or all components of the computer system may be used in conjunction with the invention. However, one of ordinary skill in the art will appreciate that other system configurations may also be used in conjunction with the invention.

In preferred embodiments, the invention is implemented on a mobile computing device, for example, a cell phone or smartphone or a tablet computing device. As used herein, a smart phone or tablet computing device refers to a multifunction mobile computing device.

The mobile multi-function device can include the circuitry of a mobile communication device that can perform some or all necessary operations. The mobile multi-function device includes hardware and software components to provide functions including media display functions, a wireless communications function, and various computing functions.

A mobile multi-function device may include a processor that pertains to a microprocessor or controller for controlling the overall operation of the mobile multi-function device. The mobile multi-function device may store media data pertaining to media items in a file system and a cache. In one embodiment, the file system is implemented by a storage disk or a plurality of disks. In another embodiment, the file system is implemented by EEPROM or flash type memory. The file system typically provides high capacity storage capability for the mobile multi-function device. The mobile multi-function device also may include RAM and Read-Only Memory (ROM). The ROM can store programs, utilities, or processes to be executed in a non-volatile manner. The ROM can be implemented by an EEPROM or Flash type memory to provide writable non-volatile data storage. The RAM provides volatile data storage, such as for a cache.

In one embodiment, to support wireless voice communications, the mobile multi-function device includes a transceiver. The transceiver supports wireless communication with a wireless network (such as a wireless cellular network or WiFi).

The mobile multi-function device may also include a user input device that allows a user of the mobile multi-function device to interact with the mobile multi-function device. For example, the user input device can take a variety of forms, such as a button, keypad, dial, etc. Still further, the mobile multi-function device includes a display (screen display) that can be controlled by the processor to display information to the user. The user input device can also be implemented as a touch-sensitive device or touchscreen apart or integral with the display. A data bus can facilitate data transfer between at least the file system, the cache, and one or more processors.

The mobile multi-function device can also include a bus interface that couples to a data link. The data link can allow the mobile multi-function device to couple to a host device (e.g., host computer or power source). The data link can also provide power to the mobile multi-function device.

The mobile electronic device utilized herein can be a hand-held electronic device. The term hand-held generally means that the electronic device has a form factor that is small enough to be held and carried around in one's hands. In some cases, the hand-held electronic device is sized for placement into a pocket of a user. By being pocket-sized, the user does not have to directly carry the device and therefore the device can be taken almost anywhere the user travels (e.g., the user is not limited by carrying a large, bulky and often heavy device).

The various aspects, features, embodiments, or implementations of the invention described above can be used alone or in various combinations.

The invention is preferably implemented by software and hardware. The invention thus also includes computer readable code on a computer readable medium. The computer readable medium is any data storage device that can store data which can thereafter be read by a computer system. Examples of a computer readable medium generally include read-only memory and random-access memory. More specific examples of computer readable medium are tangible and include Flash memory, EEPROM memory, memory card, CD-ROM, DVD, hard drive, magnetic tape, and optical data storage device. The computer readable medium can also be distributed over network-coupled computer systems so that the computer readable code is stored and executed in a distributed fashion.

As used herein, rheology refers to measurements of the flow of matter, primarily in a liquid or gas state, but also including soft solids or solids under conditions in which they respond with plastic flow rather than deforming elastically in response to an applied force. The liquids referred to herein may be Newtonian or non-Newtonian fluids including muds, emulsions, slurries, or any type of matter that exhibits flow, i.e., after some deformation it flows and is considered to include fluid properties.

As used herein, a rheometer or a conventional rheometer is a laboratory device used to measure the way in which a liquid, suspension, or slurry flows in response to applied forces. It is used for those fluids which cannot be defined by a single value of viscosity and therefore require more parameters to be set and measured than is the case for a viscometer. It measures the rheology of the fluid. Rheometers that control the applied shear stress or shear strain are called rotational or shear rheometers. See Rheometer, Wikipedia, The Free Encyclopedia, date of last revision: 4 Sep. 2020, date retrieved: 3 Dec. 2020, which is herein incorporated by reference.

A conventional rotational viscometer may operate at six rotational speeds ranging from 3 to 600 RPM. The rotation speeds may be 3 RPM, 6 RPM, 100 RPM, 200 RPM, 300 RPM, and 600 RPM. Other rotational speeds may also be used as well.

In preferred embodiments the fluid may be a drilling fluid including mud which encompasses fluids encountered in drilling operations, especially fluids that contain significant amounts of suspended solids, emulsified water, or oil. Mud includes all types of water-base, oil-base and synthetic-base drilling fluids.

### EXAMPLE

An example is here provided showing an input of density and funnel drain time to obtain a conventional rheometer Dial Reading at 600 RPM. An identical procedure is followed to obtain the predicted dial readings (and in centipoise) at any other speed. This illustration is for the Gradient Boosted Tree algorithm. The intercept is a value of 50.48. The parameters shown in FIG. 3 contribute their shares to the calculation. Their individual importance is signified by the length of the bars. Finally, the predicted dial reading is obtained to be 17.85 after all values in the red bars have been deducted.

While this disclosure includes specific examples, it will be apparent after an understanding of the disclosure of this application has been attained that various changes in form and details may be made in these examples without departing from the spirit and scope of the claims and their equivalents.

I claim:

1. A system for the measurement of rheological properties of a fluid comprising:
a computing device comprising memory, one or more processors, and software executable by the one or more processors, configured to:
accept input parameters comprising a fluid density and a fluid viscometer drain time; and
output one or more values corresponding to one or more rotations per minute (RPM) dial readings (and in centipoise) of a rotational rheometer.

2. The system of claim 1, wherein the computing device comprises a notebook computer, a smart phone, and/or a tablet computer.

3. The system of claim 2, wherein the computing device comprises a smart phone or a tablet computer.

4. The system of claim 1, wherein the input parameters consist of: fluid density and fluid viscometer drain time.

5. The system of claim 1, wherein the system outputs six dial readings (and in centipoise) corresponding to six dial readings from a rotational rheometer.

6. The system of claim 5, wherein the six dial readings correspond to 600 RPM, 300 RPM, 200 RPM, 100 RPM, 6 RPM, and 3 RPM.

7. The system of claim 1, wherein the system further outputs plastic viscosity, yield point, and/or apparent viscosity.

8. The system of claim 1, wherein the computing device is further configured to:
obtain a non-residual funnel time;
deduce a mass flow rate;
deploy six pre-trained boosted trees; and
deploy bootstrap forests to calculate the one or more RPM dial readings (and in centipoise) of a conventional rheometer.

9. The system of claim 1, wherein the computing device graphically displays input density, input drain time, and dial readings corresponding to dial readings of a conventional rotational rheometer.

10. The system of claim 1, wherein the computing device graphically displays plastic viscosity, yield point, and/or apparent viscosity.

11. The system of claim 1, wherein the fluid is a drilling fluid.

12. The system of claim 1, wherein the fluid is a mud.

13. A system for the measurement of rheological properties of a fluid comprising:
a mobile computing device comprising a smart phone or a tablet computer, comprising memory, one or more processors, and software executable by the one or more processors, configured to:
accept input parameters consisting of a fluid density and a fluid viscometer drain time; and
output and display six values corresponding to rotations per minute (RPM) dial readings (and in centipoise) of a rotational rheometer including 600 RPM, 300 RPM, 200 RPM, 100 RPM, 6 RPM, and 3 RPM.

14. The system of claim 13, further comprising outputting and displaying plastic viscosity, yield point, and/or apparent viscosity of the fluid.

15. The system of claim 13, wherein the fluid is a drilling fluid.

16. The system of claim 13, wherein the fluid is a mud.
